(19) United States
(12) Patent Application Publication
(10) Pub. No.: US 2025/0049317 A1
(43) Pub. Date: Feb. 13, 2025

(54) PRESERVING POLARIZATION MAINTAINING PHOTONS FOR ENHANCED CONTRAST IMAGING OF THE RETINA

(71) Applicant: The Board of Trustees of the University of Illinois, Urbana, IL (US)

(72) Inventors: Xincheng Yao, CHICAGO, IL (US); TaeYun Son, CHICAGO, IL (US); Alfa Rossi, CHICAGO, IL (US); Robison Chan, CHICAGO, IL (US)

(21) Appl. No.: 18/800,588

(22) Filed: Aug. 12, 2024

Related U.S. Application Data
(60) Provisional application No. 63/532,147, filed on Aug. 11, 2023.

(51) Int. Cl. A61B 3/12; A61B 3/14

(52) U.S. Cl. CPC A61B 3/12; A61B 3/14

(57) ABSTRACT

Various examples are provided related to enhanced contrast imaging of a retina. In one example, a fundus camera includes an imaging sensor; an ophthalmic lens; a quarter waveplate positioned on a side of the ophthalmic lens opposite a camera lens; a source of linearly polarized input light; and a linear polarizer disposed between the ophthalmic lens and the camera lens. The source can direct the linearly polarized input light to the quarter waveplate to convert the linearly polarized input light to circularly polarized input light that illuminates a retina. The linear polarizer can receive depolarized output light from the retina and linearly polarized output light converted by the quarter waveplate from helically flipped output light from the retina, and allow polarized output light aligned with the linear polarizer to reach the imaging sensor to provide high contrast fundus images of the retina. The fundus camera can be portable.

### CROSS REFERENCE TO RELATED APPLICATIONS

This application claims priority to, and the benefit of, U.S. Provisional Application entitled “Preserving Polarization Maintaining Photons for Enhanced Contrast Imaging of the Retina” having Ser. No. 63/532,147, filed Aug. 11, 2023, which is hereby incorporated by reference in its entirety.

### STATEMENT REGARDING FEDERALLY SPONSORED RESEARCH OR DEVELOPMENT

This invention was made with government support under awards R01 EY029673 and R44 EY028786 from the National Eye Institute (NEI); Research to Prevent Blindness; Richard and Loan Hill Endowment. The Government has certain rights in the invention.

### BACKGROUND

Ocular diseases continue to pose a significant threat to eye health worldwide, as it is projected that around 560 million people will be affected by three major retinal diseases, namely diabetic retinopathy (DR), glaucoma, and age-related macular degeneration (AMD) by the year 2045. Early detection of these diseases is enhanced with comprehensive observation of both the peripheral and the central part of the fundus and retina, which could be achieved by using modern scanning laser ophthalmoscopy (SLO) devices. Despite providing ultra-widefield images, commercially available SLO devices can be expensive, and the size of the hardware makes it challenging to deploy in remote areas.

The majority of people worldwide who are blind or have moderate to severe visual impairment (MSVI) reside in low- and middle-income countries. A significant portion of that could have been prevented by screening programs that utilize fundus imaging. Therefore, developing cost-effective, portable wide-field fundus imaging devices is important as screening programs are implemented for diabetic retinopathy, age-related macular degeneration, glaucoma, and other potentially blinding eye conditions.

### SUMMARY

Aspects of the present disclosure are related to enhanced contrast imaging of a retina. In one aspect, among others, a fundus camera comprises: a camera lens; an imaging sensor positioned on a first side of the camera lens; an ophthalmic lens positioned on a second side of the camera lens; a quarter waveplate positioned on a side of the ophthalmic lens opposite the camera lens; a source of linearly polarized input light with a polarization axis in a first direction, the source configured to direct the linearly polarized input light to the quarter waveplate through the ophthalmic lens, the quarter waveplate converting the linearly polarized input light to circularly polarized input light that illuminates a retina; and a linear polarizer disposed between the ophthalmic lens and the camera lens, the linear polarizer configured to receive depolarized output light reflected or backscattered by the retina and linearly polarized output light converted by the quarter waveplate from helically flipped output light reflected by the retina, and allow polarized output light with a polarization axis in a second direction that is aligned with the linear polarizer to reach the imaging sensor through the camera lens to provide high contrast fundus images of the retina.

In one or more aspects, the linear polarizer can simultaneously block linearly polarized input light reflected by the ophthalmic lens from reaching the imaging sensor. The source of the linearly polarized input light can comprise a light source and a corresponding linear polarizer disposed between the light source and the ophthalmic lens, the light source and the camera lens positioned in a common plane with the light source offset from the camera lens by a buffer distance. The buffer distance can be about 5 mm. The light source and the camera lens can be positioned in an area of the common plane having a diameter of about 16 mm. The light source can be configured to provide near infrared (NIR) light and visible light. The light source can comprise a first light emitting diode (LED) configured to provide 810 nm NIR light and a second LED configured to provide visible light with a center wavelength of 565 nm. In various aspects, indirect illumination by the source of the linearly polarized input light can provide a field of view of 101° eye-angle or 67° visual angle. The quarter waveplate can be positioned with its axis 45° angled with a transmission axis of the source of the linearly polarized input light to convert the linearly polarized input light to the circularly polarized input light. The fundus camera can be a portable fundus camera.

In another aspect, a method for enhanced contrast imaging of a retina comprises directing linearly polarized input light with a polarization axis in a first direction from a source positioned on a first side of an ophthalmic lens to a quarter waveplate positioned on a second side of the ophthalmic lens; converting, by the quarter waveplate, the linearly polarized input light to circularly polarized input light that illuminates a retina located adjacent to the quarter waveplate and opposite the ophthalmic lens; converting, by the quarter waveplate, helically flipped output light reflected by the retina to linearly polarized output light with a polarization axis in a second direction; receiving, by a linear polarizer positioned on the first side of the ophthalmic lens, the linearly polarized output light and depolarized output light reflected or backscattered by the retina, where the linear polarizer is aligned to allow polarized output light received with a polarization axis in the second direction to pass through the linear polarizer to an imaging sensor through a camera lens; and obtaining, by the imaging sensor, an image of the retina from the polarized output light. In one or more aspects, the linear polarizer can block linearly polarized light received by the linear polarizer without a polarization axis in the second direction. The method can comprise simultaneously blocking, by the linear polarizer, linearly polarized input light directed from the source with a polarization axis in the first direction and reflected by the ophthalmic lens.

In various aspects, the source of the linearly polarized input light can comprise a light source and a corresponding linear polarizer disposed between the light source and the ophthalmic lens, the light source and the camera lens positioned in a common plane with the light source offset from the camera lens by a buffer distance. The buffer distance can be about 5 mm. The light source can provide a combination of near infrared (NIR) light and visible light. The light source can comprise a first light emitting diode (LED) configured to provide 810 nm NIR light and a second LED configured to provide visible light with a center wavelength of 565 nm. The method can comprise providing a field of view of 101° eye-angle or 67° visual angle by indirectly illuminating the retina. The method can comprise generating a hot colormap of at least a portion of the image of the retina. The hot colormap can be of a color channel of the image.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A and 1B schematically illustrate an example of parallel polarization control that can be used for enhanced contrast imaging of a retina, in accordance with various embodiments of the present disclosure.

FIG. 1C is an image showing an example of a prototype fundus camera used during a session, in accordance with various embodiments of the present disclosure.

FIGS. 1D and 1E schematically illustrate examples of polarization states for orthogonal polarization control and parallel polarization control, in accordance with various embodiments of the present disclosure.

FIG. 2 includes fundus images of a retina taken with no polarization control, orthogonal polarization control and parallel polarization control, in accordance with various embodiments of the present disclosure.

FIGS. 3A and 3B are bar charts illustrating examples of mean intensity for orthogonal polarization control and parallel polarization control, in accordance with various embodiments of the present disclosure.

FIGS. 4A and 4B are fundus images of a retina taken with parallel polarization control and orthogonal polarization control, in accordance with various embodiments of the present disclosure.

FIG. 4C is a plot illustrating comparative line profiles of a region of the fundus images of FIGS. 4A and 4B along a line, in accordance with various embodiments of the present disclosure.

FIGS. 4D-4G are enlarged images and corresponding hot colormaps of regions in the fundus images of FIGS. 4A and 4B, in accordance with various embodiments of the present disclosure.

FIGS. 5A and 5B are high dynamic range fundus images of a retina taken with parallel polarization control and orthogonal polarization control, in accordance with various embodiments of the present disclosure.

FIGS. 5C and 5D are hot colormaps of regions in the fundus images of FIGS. 5A and 5B, in accordance with various embodiments of the present disclosure.

### DETAILED DESCRIPTION

Disclosed herein are various examples related to enhanced contrast imaging of a retina. Reference will now be made in detail to the description of the embodiments as illustrated in the drawings, wherein like reference numbers indicate like parts throughout the several views.

Fundus photography is indispensable for clinical detection and management of eye diseases. Orthogonal-polarization control can be used in conventional fundus imaging systems to minimize reflection artifacts. However, the orthogonal-polarization configuration also rejects the directly reflected photons, which preserve the polarization condition of incident light, from the superficial layer of the fundus, i.e., the retina, and thus reduce the contrast of retinal imaging. Herein, an example of a portable fundus camera is presented which can simultaneously perform orthogonal polarization control to reject back-reflected light from the ophthalmic lens and parallel polarization control to preserve the backscattered light from the retina which partially maintains the polarization state of the incoming light.

Several commercial portable fundus cameras are already being used by clinicians. Despite being portable and affordable, the FOV of these devices is typically around 45°-67.5° eye-angle (30°-45° visual angle), making them difficult to access the peripheral retina. To facilitate the visualization of the peripheral retina, miniaturized indirect illumination-based portable fundus cameras were proposed. The FOV of these nonmydriatic devices is around 101° eye-angle (67° visual angle) and may be extended up to 190° eye-angle (134° visual angle) with the aid of fixation targets. Both traditional and miniaturized indirect illumination-based portable fundus cameras use the pupillary path for illumination and imaging. The ophthalmic lens used in these systems reflects a significant amount of light back into the system, creating reflection artifacts in the image.

To remove these artifacts, orthogonal polarizers can be placed between the illumination path and the imaging path. In addition to removing the back-reflected light from the ophthalmic lens, this configuration can remove the reflected and scattered light from the retina which partially maintains the polarization state of the illumination, and only the depolarized photons coming back from the retina pass into the imaging sensor. It was calculated that about 90% of the reflected light from the human fundus partially maintain the polarization state of the input light in the mid-spectral range (480 nm-580 nm). While this number fluctuates between 70%-90% in other published literature, it has been shown that a large portion of the light in the green and blue region of the spectrum reflected from the retina preserves the polarization state of the input, and this decreases with the increase of wavelength, as only about 40% of the red light (600-650 nm) reflected from the retina preserves the polarization state of the input. The green and blue spectrum of light is needed to clearly visualize the retinal vasculature and the biomarkers of most retinal diseases. Therefore, eliminating polarization maintaining photons with orthogonal polarizers indeed reduce the information content in the green channel, eventually reducing the image contrast and the useful information regarding important retinal disease biomarkers.

In accordance with the principles presented herein, an example of a portable fundus camera configured with orthogonal polarization control for rejecting the back-reflected light from the ophthalmic lens, and the specularly reflected or backscattered light from the retina which fully or partially maintains the original polarization state that goes through to the imaging sensor to provide high contrast fundus images is described. Miniaturized indirect illumination can be implemented to achieve a field of view of 101° eye-angle (67° visual angle). The resultant image shows inner retinal pathology and biomarkers more clearly, compared to images taken with a conventional fundus camera, which would aid clinicians to detect retinal diseases more efficiently.

Traditionally, the visual angle has been the unit of FOV measurement for fundus cameras. Recently, widefield fundus imagers, such as Optomap SLO (Optos Inc., Marlborough, MA, USA), ICON (Neolight, Pleasanton, CA, USA), and Retcam (Natus Medical Systems, Pleasanton, CA, USA) use eye angle as the unit of FOV. To avoid unnecessary confusion, both eye and visual angle are presented every time the FOV of the system is discussed.

Exemplary methods and systems configured based on the principles herein can include an exemplary fundus camera with polarization control to preserve polarization maintaining photons for enhanced contrast imaging of the retina, for example. A portable device can utilize miniaturized indirect ophthalmoscopy illumination to achieve non-mydriatic imaging, with a snapshot field of view of about 101° eye-angle (about 67° visual-angle). Comparative analysis of retinal images acquired with a traditional orthogonal-polarization-based fundus camera from both normal and diseased eyes was conducted to validate the usefulness of the proposed methodology. The parallel polarization control for enhanced contrast in high dynamic range imaging was also validated.

### Materials and Methods

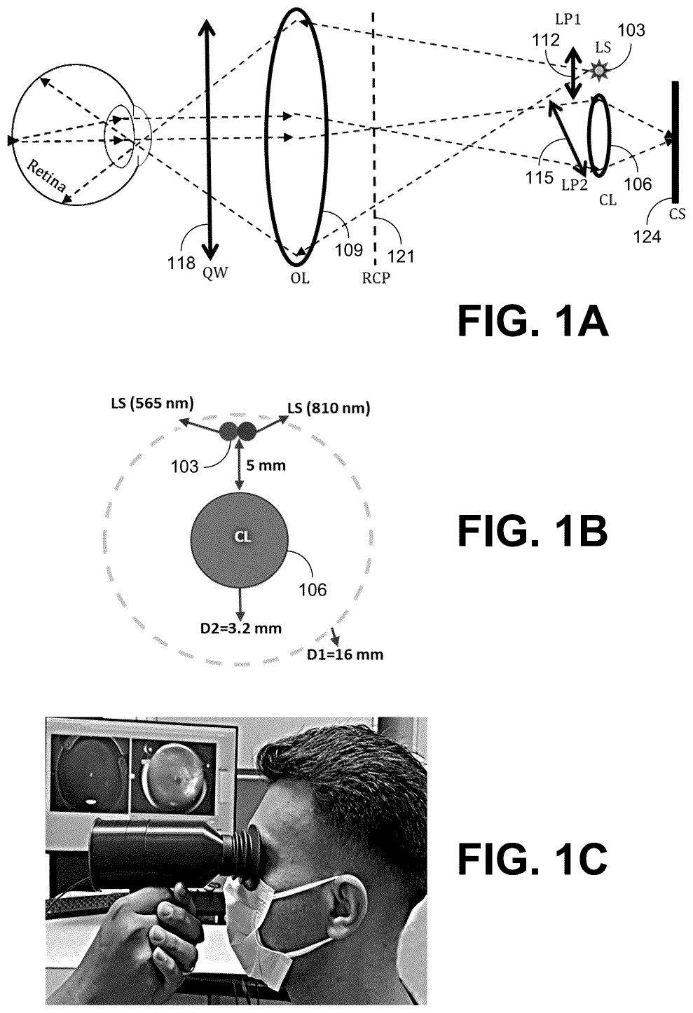

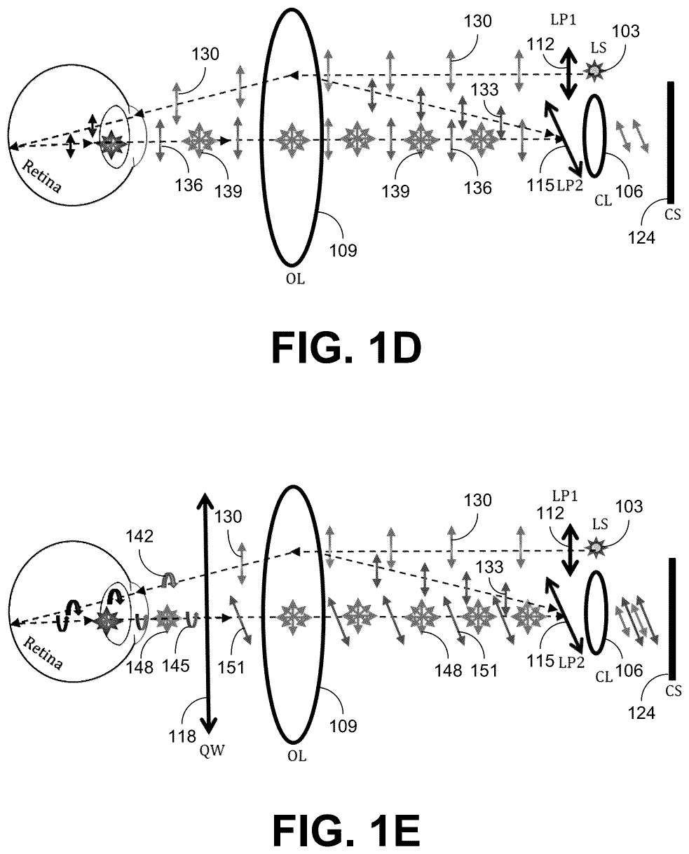

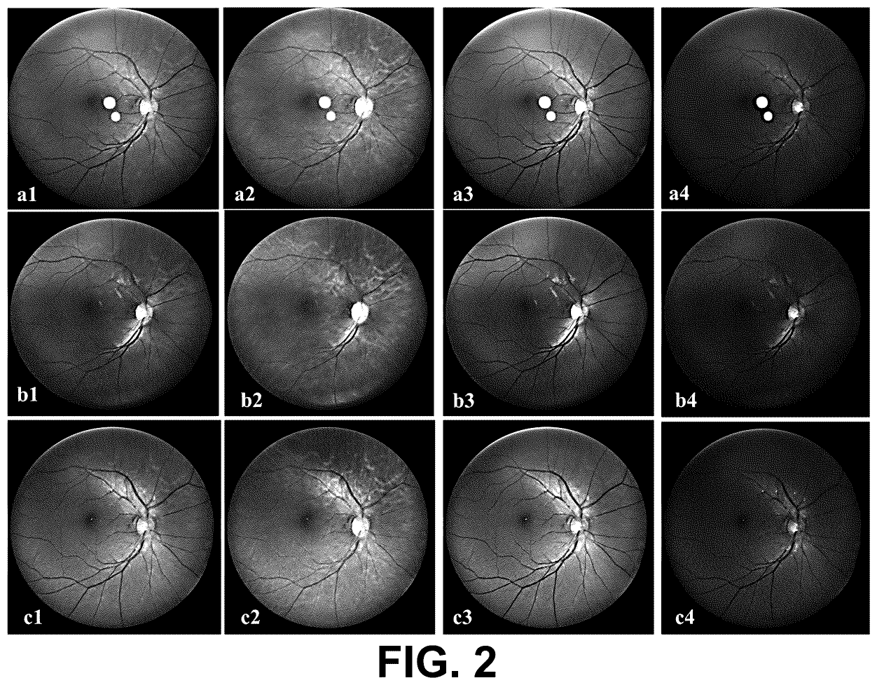

Experimental Setup. FIG. 1A is an optical diagram illustrating an example of the proposed system. The light source (LS) 103 and the camera lens (CL) 106 (e.g., 8 mm f/2.5 micro video lens, 58-203, Edmund Optics Inc., Barrington, NJ) are placed in a perpendicular plane, called the CL-LS plane. The detailed diagram of this plane is illustrated in FIG. 1B. The CL-LS plane is conjugated to the pupil plane of the eye with a magnification of 0.25× with the help of the ophthalmic lens (OL) 109 (f=25 mm). Since the diameter of the pupil is about 4 mm in room light conditions, the camera lens 106 and the light source 103 are situated inside a 16 mm diameter circle in the CL-LS plane, otherwise, the light source 103 would be blocked by the iris and no light will go inside the retina.

FIG. 1B shows that the light source 103 and the camera lens 106 are well within this limit. The light source 103 comprises two separate LEDs, an 810 nm NIR LED (e.g., M850LP1, Thorlabs Inc., Newton, NJ, USA) for guidance during initial alignment and a visible light LED with a center wavelength of 565 nm (e.g., M565D2, Thorlabs Inc., Newton, NJ, USA) for color fundus imaging. This visible light LED has a relatively wide bandwidth (FWHM 104 nm) stretching from the blue region of the light spectrum to the red end of the spectrum, which has been proven to comprehensively show retinal vasculatures and the disease biomarkers of most common retinal diseases. As shown in FIG. 1B, there is a buffer between the camera lens 106 and the light source 103 of about 5 mm, which ensures the reflected light from the corneal surface does not enter the imaging path.

Linear polarizers LP1 112 and LP2 115 (e.g., LPVISE050-A, Thorlabs Inc., Newton, NJ, USA) can be placed in front of the LS 103 and CL 106 to reject the back reflected light from OL 109. A quarter waveplate (QW) 118 (e.g., 90-940, Edmund Optics Inc., Barrington, NJ) can be placed in front of the OL 109 with its axis 45° angled with the transmission axis of LP1 112 and LP2 115 to convert the linearly polarized input light to circularly polarized one. Since the major retinal layers responsible for light reflection are independent of the polarization state of the incoming light, the absolute position of the LP1 112, LP2 115 and QW 118 is unimportant and the relative positioning is of concern here. After the light reaches the retina, an image of the retina is constructed at the retina conjugate plane (RCP) 121, and this image is relayed to the imaging or camera sensor (CS) 124 (e.g., FL3-U3-120S3C-C, FLIR Integrated Imaging Solutions Inc., Richmond, Canada) by the CL 106. To minimize the effect of eye movement and select the region of imaging, a dimly lit external fixation LED was used (not shown in the optical diagram of FIG. 1A). An imaging session with a functional prototype is shown in the image of FIG. 1C.

Preservation of the polarization maintaining photons reflected from the retina. The human retina comprises multiple functional layers and the reflected light from the retina is a complex combination of specularly reflected, diffusely reflected, and multiply scattered light coming from these layers. Out of these different layers, the outer segment (OS) layer is regarded as the primary layer responsible for specular or directional reflection which maintains its polarization state. As the light goes deeper than the OS layer and into the retinal pigment epithelium (RPE) and the choroid, it becomes depolarized because of the density and size of scatterers present in those layers.

FIGS. 1D and 1E are schematic diagrams illustrating polarization states of illumination and imaging light paths to explain the theory of preserving the polarization maintaining light reflected from the retina. FIG. 1D shows a conventional setup that works in the orthogonal-polarizing configuration. The light from the light source (LS) 103 goes through a linear polarizer (LP1) 112 and becomes linearly polarized (arrows 130 showing the direction of polarization). A portion of this light reflects from the ophthalmic lens (OL) 109 towards the camera lens 106 (marked in arrows 133) and gets blocked by the linear polarizer LP2 115. The light that goes through the ophthalmic lens 109 enters the retina, and the polarization maintaining photons (marked as arrows 136) along with depolarized photons (marked as arrows 139) come back into the imaging system. The polarization maintaining photons are blocked by the orthogonal polarizer (LP2) 115, and only the depolarized portion of the light with the polarization direction of LP2 would contribute to the image formed at the imaging sensor CS 124. Although the artifacts from the ophthalmic lens back reflection are rejected, there would be a significant loss of information as the polarization maintaining light is rejected.

FIG. 1E illustrates the proposed methodology of preserving the polarization maintaining photons coming from the retina. In the arrangement of FIG. 1E, a quarter waveplate (QW) 118 is placed in front of the ophthalmic lens 109, and the axis of the quarter waveplate 118 is fixed at an angle of 45° with the polarizer's axis. Linearly polarized input light 130 goes through the quarter waveplate 118 and becomes circularly polarized light 142 before entering the retina. Inside the retina the circularly polarized light 142 goes through the almost transparent inner-retina and the first significant reflector faced by the circularly polarized light 142 is the photoreceptor outer segment layer. The reflection from the outer segment is specular or directional in nature, therefore, the portion of the circularly polarized input light that is reflected by it is helically flipped. The remainder of the light goes into the posterior layers, gets depolarized and reflected from the choroid and the sclera. The helically flipped output photons (curved arrows 145) and the depolarized photons (arrows 148) come back into the imaging system. After going through the quarter waveplate 118 on the return path, the circularly flipped photons 145 become linearly polarized again, but their polarization axis aligns with the axis of LP2 115. Therefore, all these photons along with the depolarized photons 148 which have their polarization axis parallel to LP2 115 pass through to the imaging or camera sensor and create an image of the retina. The proposed system still works as an orthogonal polarization setup when blocking the back-reflected light from the OL 109 (marked by arrows 133). However, for convenience, it is referred to in subsequent sections as parallel polarization control since the reflected light from the retina achieves a polarization state parallel to the linear polarizer LP2 115 in front of the camera lens (CL) 106 and sensor (CS) 124.

Images taken with conventional orthogonal polarization control of FIG. 1D should be dominated by depolarized light from the deeper layer of the retina, and consequently, should be red dominant as the green light is mostly reflected from the inner retinal layers which maintains polarization. Green light that goes into the deeper layers gets absorbed and contributes very little in the image. Images taken with the parallel polarization control configuration of FIG. 1E still preserves the depolarized light reflected from the deeper layer, like the orthogonal configuration, but it also preserves the reflected light from the inner retina, providing valuable information of the disease biomarkers.

Human subjects and imaging protocol. This study was approved by the Institutional Review Board of the University of Illinois Chicago (UIC) and followed the ethical standards stated in the Declaration of Helsinki. The subjects with retinal diseases and the controls were recruited from the UIC Department of Ophthalmology & Visual Sciences clinic. The recruits consented properly before participating in the experiment. After the subjects were ready to do the imaging, the light in the room was dimmed to make sure the pupillary diameter was equal to or above 4 mm. The patients were told to keep looking at the fixation target throughout the imaging session with the eye that was not being imaged. Once the eye was fixed, NIR guidance was used to align the system with the eye without stimulating the pupillary reflex, and once it is well aligned, the images were taken with visible light. The exposure time of each acquisition was 50 ms, which is much less than the pupillary reflex time (150 ms). After taking images with the prototype using the proposed parallel polarization control methodology, the quarter waveplate 118 was removed from the prototype and the same procedure was again followed to take images for comparison. For only one of those subjects, all the polarizers were also removed from the system and an image was taken for comparison (shown in the results section below). A LABVIEW interface was designed to properly facilitate the whole imaging procedure.

Light safety. To evaluate the safety of using the prototype, the radiant exposure on the retina for visible light and the total permitted time for continuous operation for NIR guidance illumination were calculated. The safety standard was provided by ISO standard “Ophthalmic Instruments-Fundus Cameras” (10940:2009), which allowed 10 J/cm² radiant exposure on the retina. The optical power on the pupil plane for visible light and NIR light was 5 mW and 0.4 mW, respectively. Using the photochemical hazard weighting function provided in the standard, the weighted irradiance of the visible light was found, which was multiplied by the exposure time to get the radiant exposure on the retina. According to the calculation, the radiant exposure was found to be 2.4×10⁻⁶ J/cm², which was deemed safe for operation. Similarly, the weighted irradiance of NIR light was also calculated and found to be 0.06 mW/cm². The maximum permitted radiant exposure of 10 J/cm² was divided by the weighted irradiance to find out the permitted time of continuous operation. It was calculated that the NIR guidance could be used continuously for 46.3 hours.

### Results

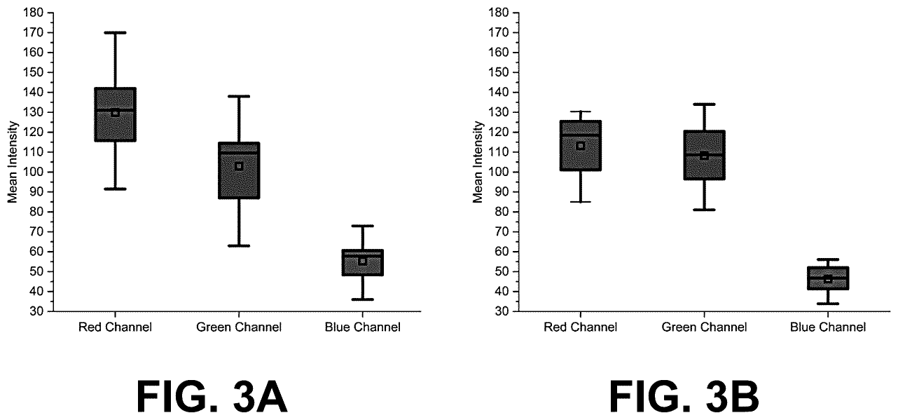

FIG. 2 illustrates fundus images of a healthy subject taken with no polarization control (top row a), orthogonal polarization control (middle row b), and the proposed parallel polarization control methodology (bottom row c). Each row includes a color fundus image (1) and separate red (2), green (3) and blue (4) channels of the fundus color image. The images taken without any polarization control (top row a) show the artifacts due to the ophthalmic lens back-reflection.

The images of FIG. 2 taken with the orthogonal polarization control (middle row b) and the parallel polarization control (bottom row c) are free from the reflection artifacts. It is evident from FIG. 2 (middle row b) that the images obtained with the orthogonal polarization control have poor vessel contrast in the color image (image b1) and the brightness of the green channel (image b3) is low. Only the major retinal blood vessels could be seen in the red channel image (image b2) in FIG. 2. Compared to that, the images in FIG. 2 obtained with the parallel polarization control (bottom row c) show overall better vessel contrast in the color image (image c1) and brightness in the green channel (image c3), painting a better picture of the retina. The red channel image (image c2) in FIG. 2 shows a similar level of choroid in addition to a lot more retinal blood vessels than image b2 in FIG. 2.

Red light can penetrate more inside the RPE and the choroid and get depolarized. The depolarized light contributes equally to each polarization state; thus, the choroidal image is similar in both images. On the other hand, the prototype also preserves the polarization maintaining red light reflected from the retina, which is proven by the improved retinal vessel contrast in the red channel image. Without any polarization control, no light reflected from the retina is rejected. Nevertheless, as most of the green light reflected from the retina maintains its polarization state, the vessel contrast is similar in the green channel images (images a2 and c2) of FIG. 2. Overall, the images taken without any polarization control look very similar to the images taken by the prototype using the parallel polarization control methodology, except for a slightly brighter choroid in the red channel image.

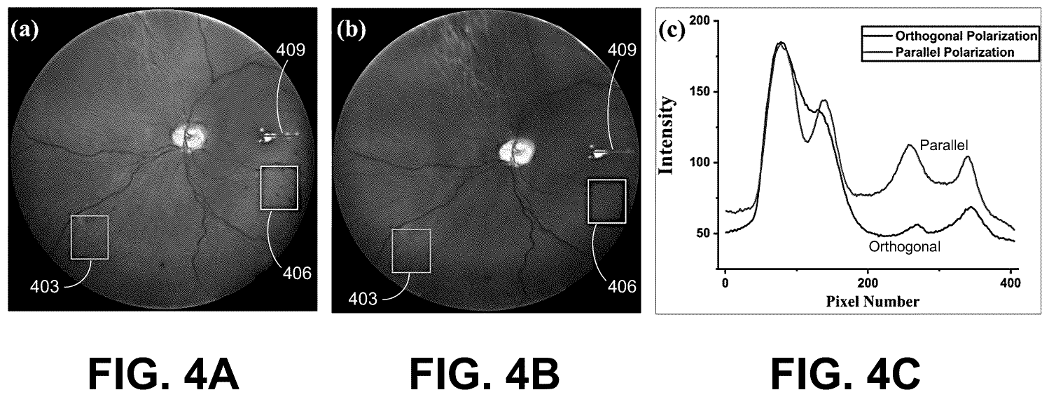

The mean intensity of the red, green, and blue channel images was calculated from ten subjects with orthogonal polarization control and the proposed parallel polarization control. The results for the orthogonal polarization control and the parallel polarization control are shown in the box plots in FIGS. 3A and 3B, respectively.

As can be seen in FIG. 3A, the mean intensity of the red channel image is much higher than the green for images taken with orthogonal polarization control. Since green light preserves more polarization maintaining photons, orthogonal polarization control rejects it more severely than red light. With parallel polarization control of FIG. 3B, the mean intensity of the red and the green channel images are almost similar, proving the preservation of polarization maintaining photons in the prototype using the parallel polarization control methodology. The blue light is strongly absorbed by the ocular lens and retinal photopigments, and mostly the optic nerve head and regions close to it can be seen in the blue channel image. As the optic nerve head also depolarizes incoming light, the mean intensity of blue channel stayed almost similar with orthogonal or parallel polarization control.

It was observed that the color fundus image taken with parallel polarization control provides better retinal vessel contrast, especially in the macular region. The red channel of the image taken with orthogonal polarization control mostly shows choroidal information due to the backscattered light with minimal retinal information. The addition of the waveplate preserves retinal layer information even in the red channel as it contains both the specularly reflected retinal layer information and the backscattered choroidal information. Similarly, retinal information was also better preserved in the green and blue channel images compared to the images acquired with orthogonal configuration. The average intensity ratio of red and green channels in the images taken with orthogonal polarization control is (1.267±0.0934):1, whereas it becomes (1.047±0.0464):1 with the parallel polarization control. As the green light is mostly reflected by the superficial retina, more photons preserve their original polarization property compared to red light with higher penetration capability. Therefore, the preservation of the polarization-maintaining photons increases the relative intensity of the green channel.

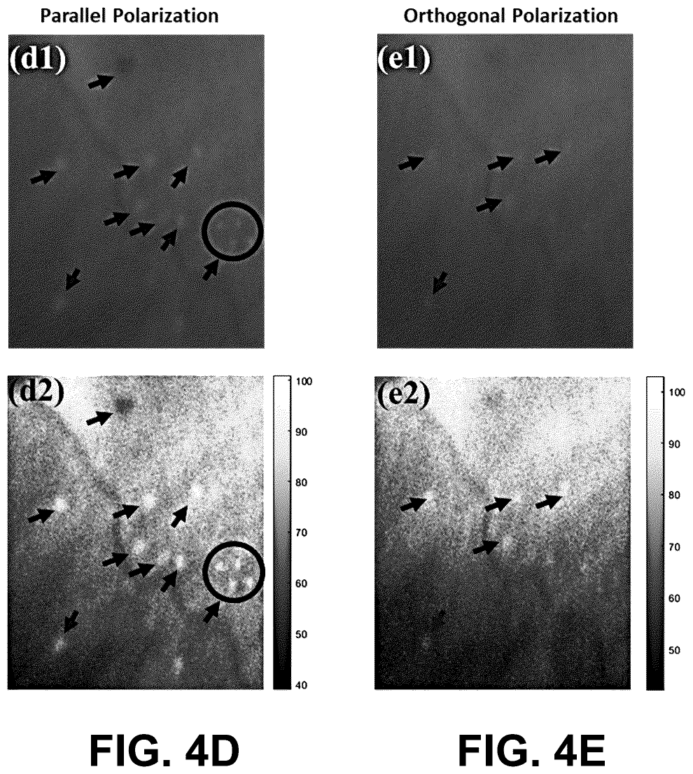

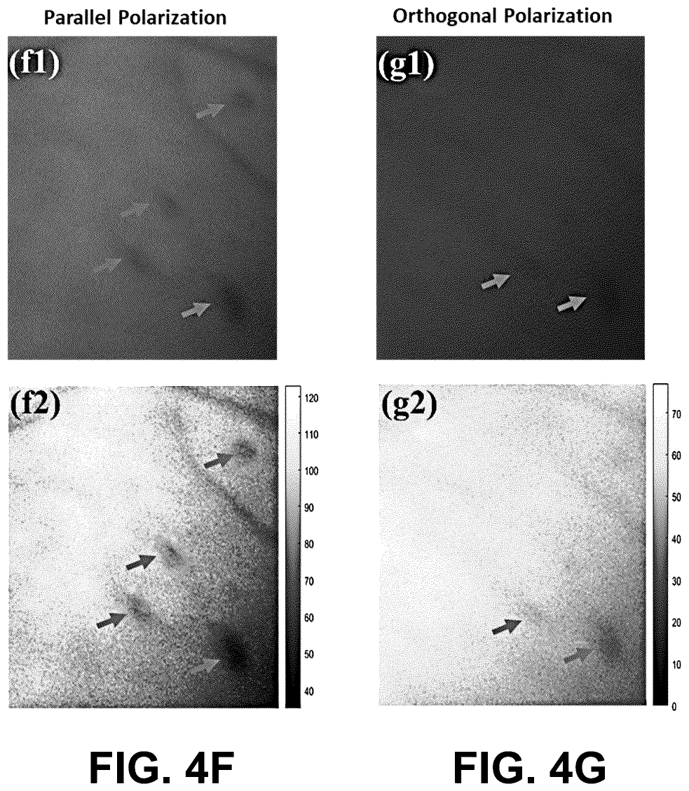

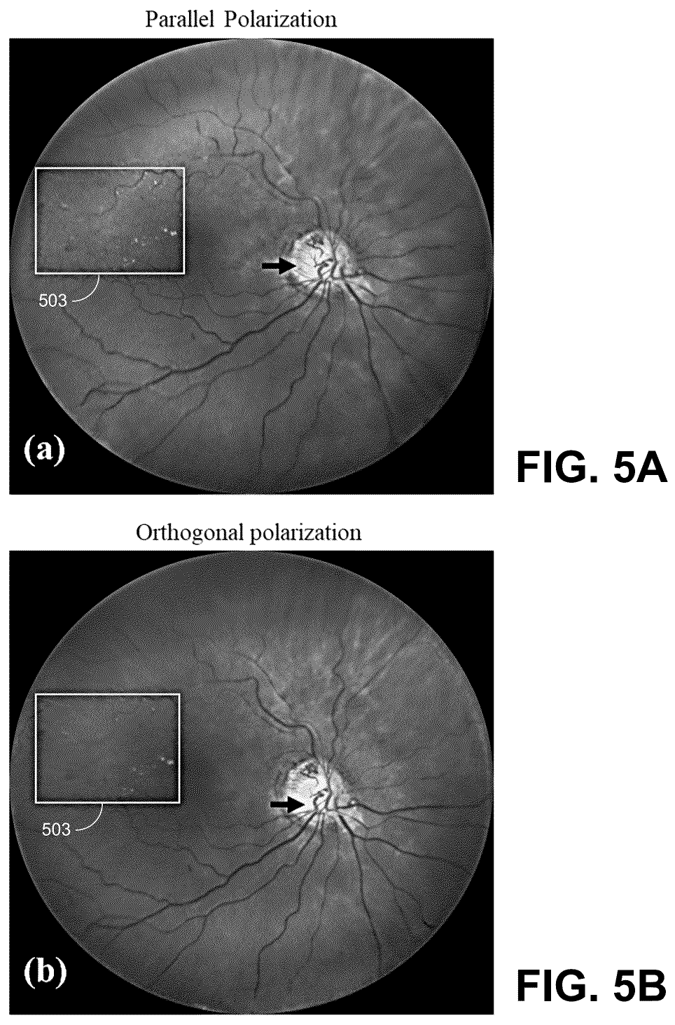

FIGS. 4A-4G illustrate the effectiveness of the parallel polarization control to detect biomarkers related to retinal diseases. FIG. 4A is an image of the retina of a diabetic retinopathy (DR) patient taken with the parallel polarization control and FIG. 4B is an image of the same patient taken with the orthogonal polarization control. From visual observation, it can be said that the image of FIG. 4A taken with the parallel polarization control has more green channel information, and thus provides more detailed information about the retina. To further investigate this, one rectangular region in the peripheral retina (marked by the rectangles 403), one rectangular region in the central retina (marked by the rectangles 406), and a region along a straight line in the fovea (marked by the line 409) were selected from both images.

FIGS. 4D and 4E show enlarged images (images d1 and e1) of the regions in rectangles 403 of FIGS. 4A and 4B, respectively, and FIGS. 4F and 4G show enlarged images (images f1 and g1) of the regions with rectangles 406 of FIGS. 4A and 4B, respectively. As the selected regions in the two images are different in brightness and tone, the corresponding hot colormaps of those regions (images d2, e2, f2 and g2) were further illustrated in FIGS. 4D-4G, respectively. The colormap negates the other factors and primarily shows the difference in the contrast. The comparative line profiles of the region along the straight lines 409 of FIGS. 4A and 4B are illustrated in the plot of FIG. 4C.

As it is visible from the enlarged images (images d1 and e1) and hot colormaps (images d2 and e2) in FIGS. 4D and 4E, the image of FIG. 4A taken with parallel polarization control significantly shows a greater number of exudates (marked with arrows) with better image contrast. Similar results can also be seen in the hot colormaps (images f2 and g2) of FIGS. 4F and 4G, where a larger number of microaneurysms (marked with arrows) can be detected in the image taken with parallel polarization control. The comparative line profiles of the foveal hard exudates (FIG. 4C) show that the parallel polarization image shows four distinct peaks of four exudates, whereas the peaks are lumped in the orthogonal polarization image. This further elucidates the enhancement of contrast by the proposed parallel polarization control methodology.

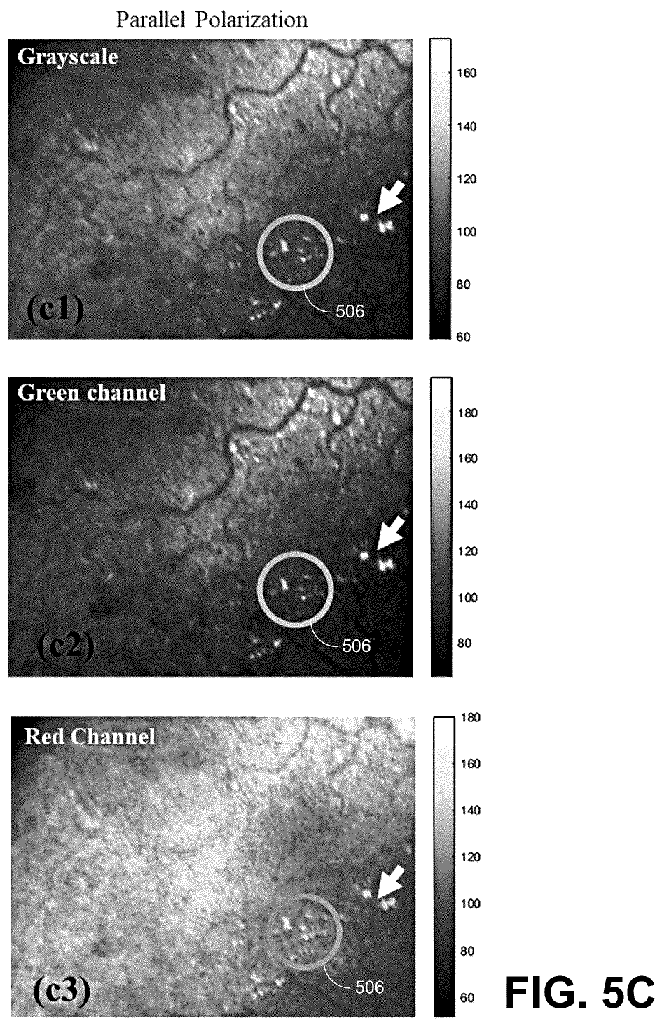

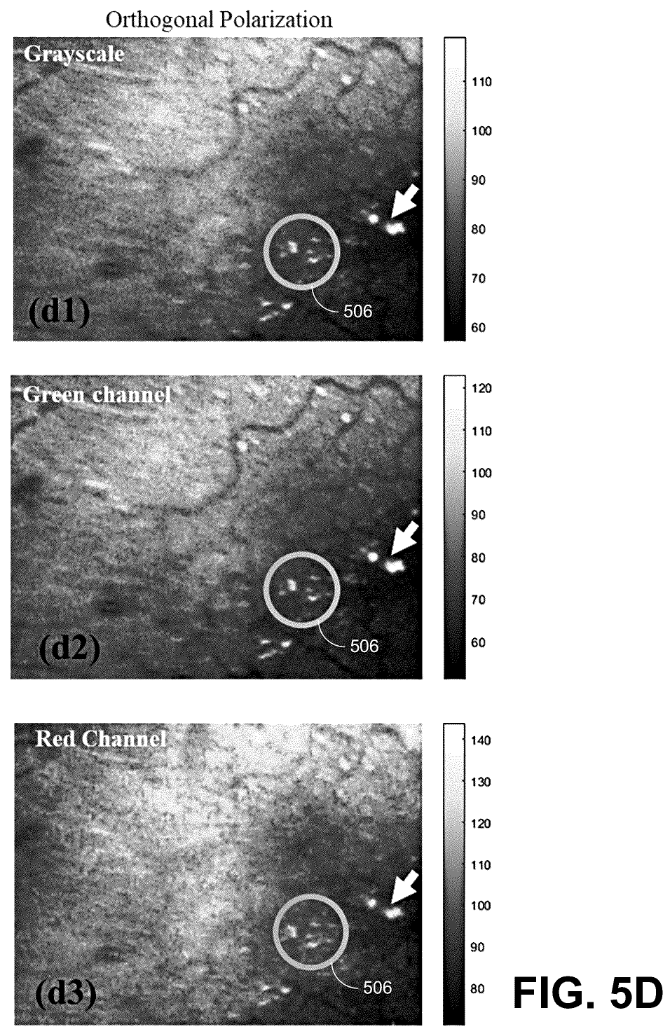

High dynamic range (HDR) imaging of the retina, coupled with orthogonal polarization control has been shown to enhance the image contrast and information content. To explore if HDR imaging with the proposed parallel polarization control can enhance the image quality even further, the procedure of HDR fundus imaging explained in previous work was followed with both the parallel and orthogonal polarization controls. FIGS. 5A and 5B illustrate two HDR images taken with parallel and orthogonal polarization control respectively, of a DR patient. From visual inspection, the optic nerve head region (marked with an arrow) in the image of FIG. 5A taken with parallel polarization control shows more information and less saturation, partly because of the preservation of the green light which is shown to capture the optic nerve head of the retina better. Furthermore, a region (marked with squares 503) was selected with exudates not far from the macula in both images.

FIGS. 5C and 5D show the hot colormaps of the grayscale images (images c1 and d1), the green channel images (images c2 and d2), and the red channel images (images c3 and d3) of the cropped region taken with parallel and orthogonal polarization control to illustrate the contrast enhancement. The Blue channel is excluded in this analysis since the information content is poor in both images for the reasons mentioned before. As can be seen, the exudates are significantly more resolved (marked with white arrows and circles 506) in the images with parallel polarization control. The green channel of the parallel polarization image shows the best contrast illustrated by the hot colormap, although the grayscale and red channel of the parallel polarization image also show better contrast of the exudates compared to their counterparts. Therefore, adopting the HDR imaging protocol in the proposed parallel polarization control system further increases the image contrast of the retina, and gives clinicians a comprehensive tool to detect and diagnose retinal diseases.

A fundus camera has been demonstrated that rejects polarization maintaining back-reflected light from the ophthalmic lens with orthogonal polarization control yet accepts the polarization maintaining light reflected from the retina with parallel polarization control. With the use of miniaturized indirect illumination, a wide FOV of 101° eye-angle (67° visual-angle) was achieved, in addition to superior image contrast than conventional portable fundus systems with orthogonal polarization control.

The retina is a layered media of different cell types superimposed onto each other with different scattering coefficients and refractive indexes of each layer. When polarized light passes through the retina, the retinal layers start to depolarize the light. However, the depolarization effect is very negligible for the inner retina before the RPE-BM complex. The light reflected and backscattered from RPE and choroid are almost completely depolarized. This applies to the light of all wavelengths. Yet, for shorter wavelengths, namely wavelengths in the green and blue region of the spectrum, mostly the directly reflected and singly scattered light returns to the imaging system. The multiply scattered photons that pass within the retina and go into the deeper layers are absorbed strongly by the melanin and choroidal pigments. As the depolarized light in the shorter wavelength is efficiently absorbed inside the retina, a significant portion (70%-90%) of the light coming back to the imaging system at least partially maintains the original polarization state. Since the absorption is weaker for longer wavelengths, about 40% of the red light returning back to the imaging path maintains its input polarization property.

The light reflected or scattered back from the retina is a superimposition of several subretinal components. Yet, multiple studies found the existence of a particular layer that reflects light almost specularly. It has been hypothesized that Bruch's membrane is the reflecting layer, whereas it has also been proposed to be somewhere near the outer segments of the photoreceptors. The outer segment (OS) layer of the photoreceptors has been pinpointed to be the specularly reflecting layer, and the anterior layers to this layer are responsible for the diffusely reflected light which also maintains the polarization partially. The layer posterior to the OS layer is responsible for the depolarized light. As retinal disease biomarkers, such as microaneurysms, cotton wool spots, exudates etc. resides in the retinal layers anterior to the OS layer, preserving the polarization maintaining light from the retina would significantly improve the detection of these biomarkers.

Polarized light has been used for selectively enhancing superficial layer images of turbid tissues. Polarization-preserving surface reflection from the air-tissue interface is one of the artifacts found in this technique. Using methods such as the usage of optically flat plate with index matching fluid and polarization gating with elliptically polarized light is used to reduce the reflection from the surface. However, for retinal imaging, the surface reflection from the inner limiting membrane (ILM)-vitreous humor interface is insignificant compared to the reflection from the OS, choroid and sclera. The reason may be attributed to the insignificant difference between the refractive index of ILM and vitreous humor, and the transparent nature of the inner retina. Nevertheless, a significant amount of surface reflection can come into the imaging system from the air-cornea interface and the lens-aqueous humor interface. The usage of para-axial illumination combined with proper buffer between the illumination and imaging window helps to prevent stray light from those abovementioned surfaces from entering the system.

An interesting property of circularly polarized light is that it can probe significantly more into scattering tissues without losing its polarization property compared to linearly polarized light when the scattering particles are bigger than the light wavelength, which is true for retinal imaging. As the illumination light becomes circularly polarized after passing the quarter waveplate in the system, the depolarization effect in the retina should be even less significant compared to linearly polarized light. Moreover, given variable orientations of the neural axons and dendrites in the retina, the depolarization effect can be locally different due to the angular complications between the local structures, such as retinal nerve fibers, and the polarization direction of a linearly polarized illumination. With circularly polarized light illumination and detection, the effect of orientational dependence of neural axons and dendrites can be minimized, and thus to increase the image homogeneity of polarization-controlled fundus photography.

Trans-pars-planar and trans-palpebral illumination in fundus imaging can provide wide FOV retinal images in a cost-effective manner. The oblique illumination also means that the image is primarily constructed by light scattered from the retina as the reflected light does not reach the imaging path. Therefore, this oblique illumination path can provide valuable complementary information if combined with the proposed system. However, the illumination light needs to pass through complex skin and scleral tissues before it reaches the retina, which makes the imaging efficiency directly affected by the illumination location, light wavelength, and subject pigmentation.

A portable, non-mydriatic, widefield fundus camera has been demonstrated that simultaneously has orthogonal polarization control to reject back reflected light from the ophthalmic lens and parallel polarization control to preserve polarization maintaining light reflected from the retina. A FOV of 101° eye-angle (67° visual angle) is achieved with the help of miniaturized indirect illumination. Comparative imaging with a conventional orthogonal polarization setup revealed the superior image quality of the proposed parallel polarization control design. This portable, high-contrast fundus camera can potentially provide clinicians in rural and under-served areas an opportunity to acquire high-quality fundus images with a wide FOV in a cost-effective manner, which will be important for telemedicine programs in ophthalmology.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. A fundus camera, comprising:
a camera lens;
an imaging sensor positioned on a first side of the camera lens;
an ophthalmic lens positioned on a second side of the camera lens;
a quarter waveplate positioned on a side of the ophthalmic lens opposite the camera lens;
a source of linearly polarized input light with a polarization axis in a first direction, the source configured to direct the linearly polarized input light to the quarter waveplate through the ophthalmic lens, the quarter waveplate converting the linearly polarized input light to circularly polarized input light that illuminates a retina; and
a linear polarizer disposed between the ophthalmic lens and the camera lens, the linear polarizer configured to receive depolarized output light reflected or backscattered by the retina and linearly polarized output light converted by the quarter waveplate from helically flipped output light reflected by the retina, and allow polarized output light with a polarization axis in a second direction that is aligned with the linear polarizer to reach the imaging sensor through the camera lens to provide high contrast fundus images of the retina.

2. The fundus camera of claim 1, wherein the linear polarizer simultaneously blocks linearly polarized input light reflected by the ophthalmic lens from reaching the imaging sensor.

3. The fundus camera of claim 1, wherein the source of the linearly polarized input light comprises a light source and a corresponding linear polarizer disposed between the light source and the ophthalmic lens, the light source and the camera lens positioned in a common plane with the light source offset from the camera lens by a buffer distance.

4. The fundus camera of claim 3, wherein the buffer distance is about 5 mm.

5. The fundus camera of claim 3, wherein the light source and the camera lens are positioned in an area of the common plane having a diameter of about 16 mm.

6. The fundus camera of claim 3, wherein the light source is configured to provide near infrared (NIR) light and visible light.

7. The fundus camera of claim 6, wherein the light source comprises a first light emitting diode (LED) configured to provide 810 nm NIR light and a second LED configured to provide visible light with a center wavelength of 565 nm.

8. The fundus camera of claim 1, wherein indirect illumination by the source of the linearly polarized input light provides a field of view of 101° eye-angle or 67° visual angle.

9. The fundus camera of claim 1, wherein the quarter waveplate is positioned with its axis 45° angled with a transmission axis of the source of the linearly polarized input light to convert the linearly polarized input light to the circularly polarized input light.

10. The fundus camera of claim 1, wherein the fundus camera is a portable fundus camera.

11. A method for enhanced contrast imaging of a retina, comprising:
directing linearly polarized input light with a polarization axis in a first direction from a source positioned on a first side of an ophthalmic lens to a quarter waveplate positioned on a second side of the ophthalmic lens;
converting, by the quarter waveplate, the linearly polarized input light to circularly polarized input light that illuminates a retina located adjacent to the quarter waveplate and opposite the ophthalmic lens;
converting, by the quarter waveplate, helically flipped output light reflected by the retina to linearly polarized output light with a polarization axis in a second direction;
receiving, by a linear polarizer positioned on the first side of the ophthalmic lens, the linearly polarized output light and depolarized output light reflected or backscattered by the retina, where the linear polarizer is aligned to allow polarized output light received with a polarization axis in the second direction to pass through the linear polarizer to an imaging sensor through a camera lens; and
obtaining, by the imaging sensor, an image of the retina from the polarized output light.

12. The method of claim 11, wherein the linear polarizer blocks linearly polarized light received by the linear polarizer without a polarization axis in the second direction.

13. The method of claim 11, comprising simultaneously blocking, by the linear polarizer, linearly polarized input light directed from the source with a polarization axis in the first direction and reflected by the ophthalmic lens.

14. The method of claim 11, wherein the source of the linearly polarized input light comprises a light source and a corresponding linear polarizer disposed between the light source and the ophthalmic lens, the light source and the camera lens positioned in a common plane with the light source offset from the camera lens by a buffer distance.

15. The method of claim 14, wherein the buffer distance is about 5 mm.

16. The method of claim 14, wherein the light source provides a combination of near infrared (NIR) light and visible light.

17. The method of claim 16, wherein the light source comprises a first light emitting diode (LED) configured to provide 810 nm NIR light and a second LED configured to provide visible light with a center wavelength of 565 nm.

18. The method of claim 11, comprising providing a field of view of 101° eye-angle or 67° visual angle by indirectly illuminating the retina.

19. The method of claim 11, comprising generating a hot colormap of at least a portion of the image of the retina.

20. The method of claim 19, wherein the hot colormap is of a color channel of the image.
